(19) United States
(12) Patent Application Publication
(10) Pub. No.: US 2017/0087068 A1
(43) Pub. Date: Mar. 30, 2017

(54) FOAMING HAIR CARE COMPOSITION

(71) Applicant: The Procter & Gamble Company, Cincinnati, OH (US)

(72) Inventors: Cedric Kofi Aurelien Callens, Singapore (SG); Stefano Bartolucci, Singapore (SG); Toshiyuki Iwata, Singapore (SG)

(21) Appl. No.: 15/274,226

(22) Filed: Sep. 23, 2016

Related U.S. Application Data
(60) Provisional application No. 62/234,038, filed on Sep. 29, 2015.

(51) Int. Cl. A61K 8/34; A61K 8/42; A61K 8/89; A61K 8/41; A61Q 5/12; A61K 8/81

(52) U.S. Cl. CPC A61K 8/342; A61Q 5/12; A61K 8/81; A61K 8/89; A61K 8/416; A61K 8/42; A61K 2800/594; A61K 2800/49; A61K 2800/5424

(57) ABSTRACT

Disclosed is a hair care composition comprising: a high melting point fatty compound; a cationic surfactant system; an aqueous carrier; and a propellant; wherein the composition has a foam density of at least about 0.3, and wherein the composition has a foam collapse resistance value of at least about 1.5 kg·m·s⁻² [N]. This foaming hair care composition provides improved wet conditioning, spreadability and/or even deposition.

### FIELD OF THE INVENTION

The present invention relates to a hair care composition comprising: a high melting point fatty compound; a cationic surfactant system; an aqueous carrier; and a propellant; wherein the composition has a foam density of at least about 0.3, and wherein the composition has a foam collapse resistance value of at least about 1.5 kg·m·s⁻² [N]. This foaming hair care composition provides improved wet conditioning, spreadability and/or even deposition.

### BACKGROUND OF THE INVENTION

Human hair becomes soiled due to its contact with the surrounding environment and from the sebum secreted by the scalp. The soiling of hair causes it to have a dirty feel and an unattractive appearance.

Shampooing cleans the hair by removing excess soil and sebum. However, shampooing can leave the hair in a wet, tangled, and generally unmanageable state. Once the hair dries, it is often left in a dry, rough, lusterless, or frizzy condition due to removal of the hair's natural oils.

A variety of approaches have been developed to alleviate these after-shampoo problems. One approach is the application of a conditioner after shampooing.

In order to provide hair conditioning benefits after shampooing, a wide variety of hair care compositions have been proposed in a variety of product forms such as liquid, oil, creams, gels, emulsions, foams/mousses and sprays.

As for foams/mousses, for example, WO2007/010487 discloses an aerosol composition characterized in that it comprises: (A) 0.1 to 5% by weight of a cationic surfactant, (B) 0.1 to 10% by weight of a fatty alcohol, (C) 0.1 to 10% by weight of carbon dioxide and (D) 0 to 1% by weight of at least one further propellant, and such aerosol compositions are said to provide creamy and rich texture. WO2007/010487 also discloses such aerosol compositions in Examples comprising 0.4-0.7% of cationic surfactants, 1.8-2.2% of fatty alcohols, and 1.5-2.5% of carbon dioxide.

Another example of foams/mousses can be EP2535037 disclosing that the objective of the invention is to provide a container with at least one inner bag, e.g., a bag-on-valve-system, in an outer container, containing a stable foam, especially a rich, thick and creamy mousse such as an aerosol cream mousse as described in WO 2007/010487. EP2535037 also discloses a composition in Example comprising 0.4% of cetrimonium chloride, 1.8% of cetearyl alcohol, and 2.0% of carbon dioxide.

However, it has been found that such foams/mousses may not be entirely satisfactory to consumers, especially in view of wet conditioning, spreadability and/or even deposition.

Based on the foregoing, there is still a need for foam/mousse hair care compositions to provide improved wet conditioning, spreadability and/or even deposition.

### SUMMARY OF THE INVENTION

The present invention is directed to a hair care composition comprising:

a high melting point fatty compound;
a cationic surfactant system;
an aqueous carrier; and
a propellant; and
wherein the composition has a foam density of at least about 0.3, and wherein the composition has a foam collapse resistance value of at least about 1.5 kg·m·s⁻² [N].

The foaming hair care composition of the present invention provides wet conditioning, spreadability and/or even deposition. The inventors of the present invention have found that the existing hair conditioning foams/mousses tend to collapse too fast and/or unevenly with or without shearing by hands, and thus provide reduced wet conditioning, spreadability and/or even deposition. Thus, the inventors of the present invention have achieved wet conditioning, spreadability and/or even deposition by the composition having a specific foam collapse resistance value.

The inventors of the present invention have also found that the composition having a specific foam density can provide improved foam collapse resistance value, and provide improved wet conditioning, spreadability and/or even deposition.

### DETAILED DESCRIPTION OF THE INVENTION

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

The term “comprising,” as used herein, means that other steps and other ingredients which do not affect the end result can be added. This term encompasses the terms “consisting of” and “consisting essentially of.” The compositions and methods/processes of the present invention can comprise, consist of, and consist essentially of the elements and limitations of the invention described herein, as well as any of the additional or optional ingredients, components, steps, or limitations described herein.

The terms “include,” “includes,” and “including,” as used herein, are meant to be nonlimiting and are understood to mean “comprise,” “comprises,” and “comprising,” respectively.

The test methods disclosed in the Test Methods Section of the present application should be used to determine the respective values of the parameters of Applicants' inventions.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated. The term “weight percent” may be denoted as “wt. %” herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Foam Density

The composition of the present invention has a foam density of at least about 0.3, preferably at least about 0.32, more preferably at least about 0.35. The foam density is preferably to about 1.2, more preferably to about 1.1, and still more preferably to about 1.0. The foam density herein is a density of the composition as dispensed. The foam density herein is the ratio of the volumetric mass of the foam to the volumetric mass of water [1 g/mL] as the reference material.

When the foam density is too low, the composition also tends to provide reduced spreadability as the foam collapses too fast to apply desired portions of the hair, and the composition may provide reduced wet conditioning benefits such as wet detangling. When the foam density is too high, the composition may lead to uneven deposition, and thus may lead the composition to provide greasiness similar to cream product.

Preferably, the composition of the present invention has the foam density of at least about 0.3 for a period of at least about 10 seconds, more preferably at least about 20 seconds, still more preferably at least about 30 seconds, in view of providing improved wet conditioning, spreadability and/or even deposition.

### Foam Collapse Resistance Value

The composition of the present invention has a foam collapse resistance value of at least about 1.5 kg·m·s⁻² [N], preferably at least about 1.7 kg·m·s⁻² [N], more preferably at least about 1.9 kg·m·s⁻² [N], in view of providing improved wet conditioning, spreadability and/or even deposition. Preferably, the foam collapse resistance value is to about 6.0 kg·m·s⁻² [N], more preferably to about 5.5 kg·m·s⁻² [N], still more preferably to about 5.0 kg·m·s⁻² [N].

The foam collapse resistance value is a resistance value of the foaming composition to prevent its macro structure from collapsing when putting a fixed load under compressed action on the foaming composition.

The foam collapse resistance values herein are measured by the following steps:

Preparing a cylindrical vessel having an inner diameter of 29 mm and a depth of 35 mm; Preparing a load having a diameter which fits to the inner diameter of the vessel, and a thickness of minimum 5 mm to avoid any unbalanced position during compression and having a weight of maximum 2.50 g;
Filling a foaming composition in the vessel to make the vessel full and scrapping any excess before putting the load on the foaming composition;
Putting the load on the foaming composition;
Measuring resistance values while the load goes down at a speed of 10 mm per minute; and
Calculate an average resistance value when the load goes down from 30% to 70% of the depth of the vessel to minimize and rule out any artifacts.

The vessel and the load can be made by any conventional materials such as plastics, preferably plastics such as thermoplastics (PLA, ABS) to avoid any additional weight added to by the compression load disk.

When the foam collapse resistance value is too low, the composition tends to provide reduced spreadability as the foam collapses too fast to apply desired portions of the hair, and the composition may provide reduced wet conditioning benefits such as wet detangling. When the foam collapse resistance value is too high, the composition tends to provide poor adhesion to hair fibers, leading to poor deposition on hair and may provide poor wet conditioning such as wet detangling.

### Cationic Surfactant System

The composition of the present invention preferably comprises a cationic surfactant system. The cationic surfactant system can be one cationic surfactant or a mixture of two or more cationic surfactants. Preferably, the cationic surfactant system is selected from: mono-long alkyl quaternized ammonium salt; a combination of mono-long alkyl quaternized ammonium salt and di-long alkyl quaternized ammonium salt; mono-long alkyl amidoamine; a combination of mono-long alkyl amidoamine and di-long alkyl quaternized ammonium salt.

The cationic surfactant system can be included in the composition at a level by weight of from about 0.1% to about 10%, preferably from about 0.5% to about 8%, more preferably from about 0.8% to about 5%, still more preferably from about 1.0% to about 4%.

### Mono-Long Alkyl Quaternized Ammonium Salt

The mono-long alkyl quaternized ammonium salt cationic surfactants useful herein are those having one long alkyl chain which has from 12 to 30 carbon atoms, preferably from 16 to 24 carbon atoms, more preferably C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

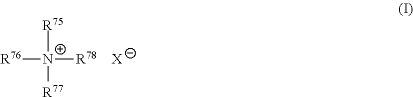

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

### Mono-Long Alkyl Amidoamine

Mono-long alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethyl amine, palmitamidoethyldiethylamine, palmitamidoethyldimethyl amine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide. Useful amines in the present invention are disclosed in U.S. Pat. No. 4,275,055, Nachtigal, et al.

These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1. In the present invention, the amounts of these acids are not included in the amount of the cationic surfactant system, and also not included in any weight or mole ratios using the cationic surfactant system.

### Di-Long Alkyl Quaternized Ammonium Salt

Di-long alkyl quaternized ammonium salt is, when used in the composition, preferably combined with a mono-long alkyl quaternized ammonium salt or mono-long alkyl amidoamine. It is believed that such combination can provide easy-to rinse feel, compared to single use of a mono-long alkyl quaternized ammonium salt or mono-long alkyl amidoamine. In such combination with a mono-long alkyl quaternized ammonium salt or mono-long alkyl amidoamine, the di-long alkyl quaternized ammonium salts are used at a level such that the wt % of the di-long alkyl quaternized ammonium salt in the cationic surfactant system is in the range of preferably from about 10% to about 50%, more preferably from about 30% to about 45%.

The di-long alkyl quaternized ammonium salt cationic surfactants useful herein are those having two long alkyl chains having 12-30 carbon atoms, preferably 16-24 carbon atoms, more preferably 18-22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

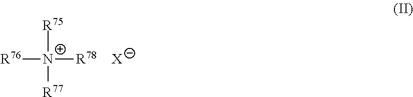

wherein two of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Such di-long alkyl quaternized ammonium salt cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride. Such dialkyl quaternized ammonium salt cationic surfactants also include, for example, asymmetric dialkyl quaternized ammonium salt cationic surfactants.

### High Melting Point Fatty Compound

The high melting point fatty compound can be included in the composition at a level of from about 0.5% to about 20%, preferably from about 1% to about 15%, more preferably from about 1.5% to about 8%, still more preferably from about 2.5% to about 7%, even more preferably from about 3.0% to about 6% by weight of the composition, in view of providing improved foam properties and improved conditioning benefits by lamellar foam structure.

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

### Aqueous Carrier

The composition of the present invention preferably comprises an aqueous carrier. The level and species of the carrier are selected according to the compatibility with other components, and other desired characteristic of the product.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 30% to about 95%, and more preferably from about 80% to about 95% water.

### Propellant

The composition of the present invention comprises a propellant. The propellant can be any materials that are known in the art as propellants, and for example, trichlorofluoromethane, dichlorodifluoromethane, dichlorotetrafluoroethane, monochlorodifluoromethane, trichlorotrifluoroethane, dimethylether, carbon dioxide, and hydrocarbons such as propane, n-butane and isobutane, and mixtures thereof. The hydrocarbons, particularly isobutane, used singly or admixed with other hydrocarbons, are preferred due to their densities being less than 1.

The propellant is contained in the composition at a level of preferably from about 1% to about 15%, more preferably from about 2% to about 10%, still more preferably from about 2.5% to about 5.5% by weight of the composition. When the propellant such as dimethylether utilizes a vapor pressure suppressant (e.g., trichloroethane or dichloromethane), the amount of suppressant is included as part of the propellant.

### Gel Matrix

Preferably, in the present invention, a gel matrix is formed by the cationic surfactant, the high melting point fatty compound, and an aqueous carrier. When such gel matrix is contained, the discrete particles of the oily components are dispersed in such gel matrix. The gel matrix is suitable for providing various conditioning benefits, such as slippery feel during the application to wet hair and softness and moisturized feel on dry hair.

Preferably, when the gel matrix is formed, the cationic surfactant system and the high melting point fatty compound are contained at a level such that the weight ratio of the cationic surfactant system to the high melting point fatty compound is in the range of, preferably from about 1:1 to about 1:10, more preferably from about 1:1.5 to about 1:7, still more preferably from about 1:2 to about 1:6, even more preferably from about 1:2 to about 1:4, in view of providing improved foam properties and the benefits of the present invention.

Preferably, especially when the gel matrix is formed, the total amount weight % of the cationic surfactant system and the high melting point fatty compound is from about 4.0%, more preferably from about 4.2%, still more preferably from about 4.5%, even more preferably from about 5.0%, further more preferably from about 6.0% by weight of the composition, in view of providing improved foam properties and the benefits of the present invention, and to about 15%, preferably to about 12%, more preferably to about 10%, still more preferably to about 8% by weight of the composition, in view of providing improved foam properties and the benefits of the present invention by providing desired lamellar gel network.

In the present invention, it is preferred that the cationic surfactant is included such that the mol % of the cationic surfactant to a sum of the cationic surfactant and the high melting point fatty compound is from about 18% to about 30%, more preferably from about 22% to about 28%, still more preferably from about 24% to about 27%, in view of providing improved foam properties and the benefits of the present invention. If the mol % is too low, the composition may provide more fatty alcohol crystallization and thus non-homogeneous gel matrix, leading to non-homogenous foam spreading and deposition. If the weight % is too high, the composition may provide more vesicle rather than desired lamellar sheets structure, again potentially leading to reduced wet detangling during application to hair.

Preferably, when the gel matrix is formed, the composition of the present invention is substantially free of anionic surfactants, in view of stability of the gel matrix. In the present invention, “the composition being substantially free of anionic surfactants” means that: the composition is free of anionic surfactants; or, if the composition contains anionic surfactants, the level of such anionic surfactants is very low. In the present invention, a total level of such anionic surfactants, if included, preferably 1% or less, more preferably 0.5% or less, still more preferably 0.1% or less by weight of the composition. Most preferably, the total level of such anionic surfactants is 0% by weight of the composition.

### Silicone Compound

The compositions of the present invention may contain a silicone compound. The silicone compounds are included at levels by weight of the composition of from about 0.05% to about 15%, preferably from about 0.1% to about 10%, more preferably from about 0.1% to about 6%.

Preferably, the silicone compounds have an average particle size of from about 1 microns to about 50 microns, in the composition.

The silicone compounds useful herein, as a single compound, as a blend or mixture of at least two silicone compounds, or as a blend or mixture of at least one silicone compound and at least one solvent, have a viscosity of preferably from about 1,000 to about 2,000,000 mPa·s at 25° C.

The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

In some embodiments, amino substituted silicones are preferably used. Preferred aminosilicones include, for example, those which conform to the general formula (I):

(R₁)ₐG₃₋ₐ-Si-(−OSiG₂)ₙ-(−OSiG_{b}(R₁)_{2-b})ₘ—O—SiG₃₋ₐ(R₁)ₐ

wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion.

Highly preferred amino silicones are those corresponding to formula (I) wherein m=0, a=1, q=3, G=methyl, n is preferably from about 1500 to about 1700, more preferably about 1600; and L is —N(CH₃)₂ or —NH₂, more preferably —NH₂. Another highly preferred amino silicones are those corresponding to formula (I) wherein m=0, a=1, q=3, G=methyl, n is preferably from about 400 to about 600, more preferably about 500; and L is —N(CH₃)₂ or —NH₂, more preferably —NH₂. Such highly preferred amino silicones can be called as terminal aminosilicones, as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone compounds include those having alkylamino substitutions as pendant groups of a silicone backbone. Highly preferred are those known as “amodimethicone”. Commercially available amodimethicones useful herein include, for example, BY16-872 available from Dow Corning.

### Anionic Deposition Polymer

The composition of the present invention may further comprise an anionic polymer, preferably anionic deposition polymer, in view of improving out of shower styling and manageability of hair to achieve the desired style. The deposition polymer is included at a level by weight of the composition of, from about 0.05% to about 6%, preferably from about 0.1% to about 5%, more preferably from about 0.2% to about 3.5%.

The anionic polymers useful herein are those comprising a vinyl monomer (A) with a carboxyl group, wherein the vinyl monomer (A) is contained in the polymer at a level of from about 10 mass % to 90 mass % based on the total mass of the copolymer.

Especially for anionic deposition polymers, it is preferred that the weight ratio of (i) the anionic deposition polymer to (ii) a sum of the cationic surfactant and high melting point fatty compound is from about 1:1 to about 1:160, more preferably from about 1:2.5 to about 1:120, still more preferably from about 1:3.5 to about 1:80. If the weight ratio of (i) to (ii) is too low, the composition may provide lower deposition of cationic surfactants, high melting point fatty compounds, and/or silicone compounds. If the weight ratio of (i) to (ii) is too high, the composition may influence rheology, and may undesirably decrease rheology of the composition.

### Anionic Deposition Polymer

The deposition polymer useful herein is a copolymer comprising: a vinyl monomer (A) with a carboxyl group in the structure; and a vinyl monomer (B) expressed by the following formula (1):

CH₂═C(R¹)—CO—X-(Q-O)ᵣ—R² (1)

wherein: R¹ represents a hydrogen atom or a methyl group; R² represents a hydrogen atom or an alkyl group with from 1 to 5 carbon atoms, which may have a substitution group; Q represents an alkylene group with from 2 to 4 carbon atoms which may also have a substitution group; r represents an integer from 2 to 15; and X represents an oxygen atom or an NH group; and, in the following structure -(Q-O)ᵣ—R², the number of atoms bonded in a straight chain is 70 or less; and
wherein the vinyl monomer (A) is contained at a level of from about 10 mass % to about 90 mass %, and the vinyl monomer (B) is contained at level of from about 10 mass % to about 90 mass %.

### Vinyl Monomer (A)

The copolymer of the present invention contains a vinyl monomer (A) having a carboxyl group in the structure. The copolymer may contain one type of the vinyl monomer (A), or may contain two or more types of the vinyl monomer (A). The vinyl monomer (A) is preferably anionic.

Non-limited example of the vinyl monomer (A) having a carboxyl group include, for example, unsaturated carboxylic acid monomers having 3 to 22 carbon atoms. The unsaturated carboxylic acid monomer has, preferably 4 or more carbon atoms, and preferably 20 or less carbon atoms, more preferably 18 or less carbon atoms, still more preferably 10 or less carbon atoms, and even more preferably 6 or less carbon atoms. Furthermore, the number of carboxyl groups in the vinyl monomer (A) is preferably from 1 to 4, more preferably from 1 to 3, even more preferably from 1 to 2, and most preferably 1.

In view of improved deposition of cationic surfactants, fatty compounds and/or silicones, the vinyl monomer (A) is preferably an unsaturated carboxylic acid monomer expressed by the following formula (2) or formula (3), more preferably those expressed by the formula (2)

CH₂═C(R³)—CO—(O—(CH₂)ₘ—CO)ₙ—OH (2)

wherein: R³ represents a hydrogen atom or a methyl group, preferably a hydrogen atom; m represents an integer of 1 through 4, preferably 2 to 3; and n represents an integer of 0 through 4, preferably 0 to 2, and most preferably 0

CH₂═C(R⁴)—COO—(CH₂)p-OOC—(CH₂)q-COOH (3)

wherein: R⁴ represents a hydrogen atom or a methyl group, preferably a hydrogen atom; p and q independently represent an integer of 2 through 6, preferably 2 to 3.

Examples of those expressed by the formula (2) include (meth)acrylic acid, crotonic acid, maleic acid, fumaric acid, itaconic acid, angelic acid, tiglic acid, 2-carboxy ethyl acrylate oligomer, and the like. Among them, preferred are acrylic acid and methacrylic acid, and more preferred is acrylic acid. Examples of those expressed by the formula (3) include acryloyloxy ethyl succinate, 2-methacryloyloxy ethyl succinate, and the like.

### Vinyl Monomer (B)

The copolymer contains a vinyl monomer (B). The copolymer may contain one type of the vinyl monomer (B), or may contain two or more types of the vinyl monomer (B). The vinyl monomer (B) is preferably nonionic.

The Vinyl monomers (B) useful herein are those expressed by formula (4)

CH₂═C(R¹)—CO—X-(Q-O)ᵣ—R² (4)

wherein: R¹ represents a hydrogen atom or a methyl group; R² represents a hydrogen atom or an alkyl group with 1 through 5 carbon atoms, which may have a substitution group; Q represents an alkylene group with 2 through 4 carbon atoms which may also have a substitution group; r represents an integer from 2 through 15; and X represents an oxygen atom or an NH group; and in the structure -(Q-O)ᵣ—R², the number of atoms bonded in a straight chain is 70 or less.

If R² has a substitution group, the substitution group is a substitution group that does not react with other parts of the copolymer. The vinyl monomer (B) is preferably hydrophilic, and therefore R² is preferably a hydrogen atom or an alkyl group with 1˜3 carbon atoms, and more preferably a hydrogen atom or an alkyl group with 1 or 2 carbon atoms.

X preferably represents an oxygen atom.

Q represents preferably an alkylene group with 2 through 3 carbon atoms which may also have a substitution group, and more preferably an alkylene group with 2 through 3 carbon atoms without any substitution group. If the alkylene group of Q has a substitution group, it is preferred that such substitution group does not react with other parts of the copolymer, more preferably such substitution group has a molecular weight of 50 or less, still more preferably such substitution group has a molecular weight that is smaller than the structural moiety of -(Q-O)ᵣ—. Examples of such substitution group include a hydroxyl group, methoxy group, ethoxy group, and the like.

r represents preferably 3 or higher, and preferably 12 or less, in view of improved deposition of cationic surfactants, fatty compounds and/or silicones, and/or in view of smoothness during application.

As described above, in the structure -(Q-O)ᵣ—R², the number of atoms that are bonded by the straight chain is 70 or less. For example, if Q represents an n-butylene group, r=15, and R² represents an n-pentyl group, the number of atoms that are bonded in the straight chain of the structure -(Q-O)ᵣ—R² is calculated as 80, which therefore is outside of the scope. The number of atoms bonded in the straight chain in the structure -(Q-O)ᵣ—R² is preferably 60 or less, more preferably 40 or less, even more preferably 28 or less, and particularly preferably 20 or less, in view of improved deposition of cationic surfactants, fatty compounds and/or silicones, and/or in view of smoothness during application.

Examples of the vinyl monomer (B) include, methoxy polyethylene glycol (meth)acrylate (where the number of repetitions of polyethylene glycol (r in formula (4)) is between 2˜15), polyethylene glycol (meth)acrylate (where the number of repetitions of polyethylene glycol (r in formula (4)) is between 2˜15), methoxy polyethylene glycol/polypropylene glycol (meth)acrylate (where the number of repetitions of polyethylene glycol/polypropylene glycol (r in formula (4)) is between 2˜15), polyethylene glycol/polypropylene glycol (meth)acrylate (where the number of repetitions of polyethylene glycol/polypropylene glycol (r in formula (4)) is between 2˜15), methoxy polyethylene glycol/polybutylene glycol (meth)acrylate (where the number of repetitions of polyethylene glycol/polybutylene glycol (r in formula (4)) is between 2˜15), polyethylene glycol/polybutylene glycol (meth)acrylate (where the number of repetitions of polyethylene glycol/polybutylene glycol (r in formula (4)) is between 2˜15), methoxy polyethylene glycol (meth)acrylamide (where the number of repetitions of polyethylene glycol (r in formula (4)) is between 2˜15), and polyethylene glycol (meth)acrylamide (where the number of repetitions of polyethylene glycol (r in formula (4)) is between 2˜15); preferably methoxy polyethylene glycol (meth)acrylate (where the number of repetitions of polyethylene glycol (r in formula (4)) is between 3˜12), polyethylene glycol (meth)acrylate (where the number of repetitions of polyethylene glycol (r in formula (4)) is between 3˜12), methoxy polyethylene glycol/polypropylene glycol (meth)acrylate (where the number of repetitions of polyethylene glycol/polypropylene glycol (r in formula (4)) is between 3˜12), polyethylene glycol/polypropylene glycol (meth)acrylate (where the number of repetitions of polyethylene glycol/polypropylene glycol (r in formula (4)) is between 3˜12), methoxy polyethylene glycol/polybutylene glycol (meth)acrylate (where the number of repetitions of polyethylene glycol/polybutylene glycol (r in formula (4)) is between 3˜12), polyethylene glycol/polybutylene glycol (meth)acrylate (where the number of repetitions of polyethylene glycol/polybutylene glycol (r in formula (4)) is between 3˜12); more preferably methoxy polyethylene glycol (meth)acrylate (where the number of repetitions of polyethylene glycol (r in formula (4)) is between 3˜12), and polyethylene glycol (meth)acrylate (where the number of repetitions of polyethylene glycol (r in formula (4)) is between 3˜12).

### Vinyl Monomer (C)

In addition to the vinyl monomers (A) and (B), the copolymer may further contain a vinyl monomer (C) having an alkyl group with 12˜22 carbon atoms, in view of providing conditioning effect such as smoothness during application. When included, the amount of the vinyl monomer (C) is preferably 40 mass % or less, more preferably 30 mass % or less, even more preferably 25 mass % or less, and still more preferably 20 mass % or less based on the total mass of the copolymer, in view of improved deposition of cationic surfactants, fatty compounds and/or silicones, and/or in view of smoothness during application.

Preferably, the vinyl monomer (C) is a (meth)acrylate monomer having an alkyl group with 12˜22 carbon atoms, in view of smoothness during application. Furthermore, vinyl monomers with branched alkyl groups are particularly preferred.

Examples of the (meth)acrylate monomer having an alkyl group with 12˜22 carbon atoms include myristyl (meth)acrylate, isostearyl (meth)acrylate, stearyl (meth)acrylate, behenyl (meth)acrylate, cetyl (meth)acrylate, lauryl (meth)acrylate, synthetic lauryl (meth)acrylate, (however “synthetic lauryl (meth)acrylate” refers to an alkyl (meth)acrylate having alkyl groups with 12 carbon atoms and alkyl groups with 13 carbon atoms), and the like. Of these, (meth)acrylate monomers having an alkyl group with 12˜20 carbon atoms are preferable, and (meth)acrylate monomers having an alkyl group with 16˜18 carbon atoms are more preferable.

The copolymer may contain one type of the vinyl monomer (C), or may contain two or more types of the vinyl monomer (C).

### Other Monomers

In addition to the aforementioned vinyl monomers (A), (B), and (C), the copolymer may also contain other vinyl monomers, to the extent not to deteriorate the effect of the copolymer. Examples of other vinyl monomers include nonionic monomers, amphoteric monomers, semipolar monomers, cationic monomers, as well as monomers containing a polysiloxane group, preferably nonionic monomers with or without polysiloxane group These other monomers are different from any of the aforementioned vinyl monomers (A), (B), and (C).

Normally the amount of such other monomers, if included, is 40 mass % or less of the total mass of the copolymer, preferably 30 mass % or less, more preferably 20 mass % or less, and even more preferably 10 mass % or less.

In view of improved deposition of cationic surfactants, fatty compounds, and/or silicones, the amount of cationic functional groups in the copolymer is preferably low, and for example cationic functional groups preferably account for 10 mole % or less of all functional groups in the copolymer. More preferably, the copolymer is free of cationic functional groups.

Examples of nonionic monomers include esters of (meth)acrylic acid and alcohols with 1˜22 carbon atoms, amides of (meth)acrylic acid and alkyl amines with 1˜22 carbon atoms, monoesters of (meth)acrylic acid and ethylene glycol, 1,3-propylene glycol or the like, as well as esters where the hydroxyl group of the monoester has been etherified by methanol, ethanol or the like, (meth)acryloyl morpholine and the like.

Examples of amphoteric monomers include (meth)acryl esters having a betaine group, (meth)acrylamide having a betaine group and the like.

Examples of semipolar monomers include (meth)acrylate esters having an amine oxide group, (meth)acrylamides having an amine oxide group, and the like.

Examples of cationic monomers include (meth)acrylate esters having a quaternary ammonium group, (meth)acrylamides having a quaternary ammonium group and the like.

The monomer containing a polysiloxane group is a monomer having a polysiloxane structure and also having a structure that can bond by covalent bond to the copolymer. These component units have high affinity towards silicone oil that is normally used in conjunction in cosmetic material compositions, and are thought to act by bonding the silicone oil to the other component units in the copolymer and thus increasing the adsorption force of silicone oil to the skin and hair, particularly damaged hair.

The polysiloxane structure is a structure where two or more repeating structural units expressed by the following formula (4) are linked.

—(SiR⁵R⁶—O)— (4)

In formula (4), R⁵ and R⁶ independently represent an alkyl group with 1 to 3 carbon atoms or a phenyl group.

The structure that can link via covalent bond to the copolymer can be a structure that has a vinyl structure such as a (meth)acrylate ester, or (meth)acrylamide and that can copolymerize with another monomer, a structure that has a functional group such as a thiol, that can link to the copolymer by chain transfer during polymerization, or a structure that has an isocyanate group, carboxylic acid group, hydroxyl group, amino group, or the like, and that can react and link to the functional groups on the copolymer, but there is no restriction to these structures.

A plurality of these linkable structures can be present in one monomer containing a polysiloxane group. In the copolymer, the polysiloxane structure can link by a graft structure to the main chain, or conversely the polysiloxane structure can be the main chain with the other structure link by a graft structure, and in addition the polysiloxane structure and the other structure can be linked in a straight chain condition by a block structure.

The monomer containing a polysiloxane group is preferably expressed by the following formula (5).

CH₂═C(R⁷)—Z—(SiR⁸R⁹—O)ₛ—R¹⁰ (5)

In the formula, R⁷ represents a hydrogen atom or a methyl group, R⁸ and R⁹ independently represent an alkyl group with 1 to 3 carbon atoms or a phenyl group, R¹⁰ represents an alkyl group with 1 to 8 carbon atoms, Z represents a bivalent linking group or a direct bond, and s represents an integer between 2 to 200.

More preferably, s is 3 or higher, and even more preferably, s is 5 or higher, in view of increased affinity to silicone oil, and preferably s is 50 or less, in view of enhanced copolymerization with the other monomers.

Z represents a bivalent linking group or a direct bond, but a linking group containing one or a combination of two or more of the structures suggested below is preferable. The numbers that are combined is not particularly restricted, but normally is 5 or less. Furthermore, the direction of the following structures are arbitrary (the polysiloxane group side can be on either end). Note, in the following, R represents an alkylene group with 1 to 6 carbon atoms or a phenylene group.
—COO—R—
—CONH—R—
—O—R—
—R—

The monomer expressed by the aforementioned formula (5), include, for example, α-(vinyl phenyl) polydimethyl siloxane, α-(vinyl benzyloxy propyl) polydimethyl siloxane, α-(vinyl benzyl) polymethyl phenyl siloxane, α-(methacryloyl oxypropyl) polydimethyl siloxane, α-(methacryloyloxy propyl) polymethyl phenyl siloxane, α-(methacryloyl amino propyl) polydimethyl siloxane and the like. The monomer containing a polysiloxane group can be a single type, or can be two or more types used in combination.

In order to adjust the molecular weight and the viscosity of the copolymer, a cross-linking agent such as a polyfunctional acrylate or the like can be introduced to the copolymer. However, in this invention, it is preferred that a cross-linking agent is not included in the copolymer.

### Structure Analysis

The amount of the vinyl monomers (A), (B), and (C) as well as other monomers in the copolymer can be measured using IR absorption or Raman scattering by the carbonyl groups, amide bonds, polysiloxane structures, various types of functional groups, carbon backbone and the like, by ¹H-NMR of methyl groups in the polydimethyl siloxane, amide bond sites, and methyl groups and methylene groups adjacent thereto, as well as various types of NMR represented by ¹³C-NMR and the like.

### Weighted Average Molecular Weight

The weighted average molecular weight of the copolymer is preferably about 3,000 or higher, more preferably about 5,000 or higher, and even more preferably about 10,000 or higher, in view of providing conditioning effect via foaming a complex with cationic surfactant, and preferably to about 2,000,000, more preferably about 1,000,000 or less, still more preferably about 500,000 or less, even more preferably about 100,000 or less, and most preferably about 50,000 or less, in view of feeling after drying.

The weighted average molecular weight of the copolymer can be measured by gel permeation chromatography (GPC). The development solvent that is used in gel permeation chromatography is not particularly restricted so long as being a normally used solvent, but for example, the measurement can be performed using a solvent blend of water/methanol/acetic acid/sodium acetate.

### Viscosity

The copolymer preferably has a viscosity for a 20 mass % ethanol solution at 25° C. of 5 mPa·s or higher and 20,000 mPa·s or less. The viscosity is more preferably 10 mPa·s or higher, even more preferably 15 mPa·s or higher, but on the other hand is more preferably 10,000 mPa·s or less, and even more preferably 5,000 mPa·s or less. The viscosity of the copolymer is preferably 5 mPa·s or higher and 20,000 mPa·s or less, from the perspective of handling. The viscosity can be measured using a B-type viscometer.

Similar to the weighted average molecular weight, the viscosity of the copolymer can be adjusted by controlling the degree of polymerization of the copolymer, and can be controlled by increasing or decreasing the amount of a cross-linking agent such as a polyfunctional acrylate or the like that is added.

### Soy Oligomer

The composition may further comprise a mixture of a soy oligomer and soy bean oil, in view of improving out of shower styling and manageability of hair to achieve the desired style.

The hair care composition comprises such soy oligomer at a level of from about 0.005% to about 5%, preferably from about 0.01% to about 3%, more preferably from about 0.01% to about 2%, still more preferably from about 0.01% to about 1% by weight of the hair care composition. The hair care composition comprises such soy bean oil at a level of from about 0.005% to about 20%, preferably from about 0.01% to about 15%, more preferably from about 0.03% to about 10%, still more preferably from about 0.03% to about 5%, by weight of the hair care composition. The weight ratio of the soy bean oil to the soy oligomer is preferably from about 98:2 to about 70:30, more preferably from about 95:5 to about 75:25, still more preferably from about 95:5 to about 80:20.

Oligomers useful herein include, for example, dimer, trimer, tetramer, pentamer, and/or hexamer, preferably, dimer, trimer, and/or tetramer, more preferably, a mixture of dimer, trimer, and/or tetramer. The oligomers may be further modified via hydrogenation. For example, in certain embodiments, the oligomer can be about 60% hydrogenated or more; in certain embodiments, about 70% hydrogenated or more; in certain embodiments, about 80% hydrogenated or more; in certain embodiments, about 85% hydrogenated or more; in certain embodiments, about 90% hydrogenated or more; and in certain embodiments, generally 100% hydrogenated.

### Package

The composition of the present invention can be provided with any package, such as aerosol cans. The composition of the present invention can also be provided with a package comprising at least one inner bag and an outer container as disclosed in EP2535037, wherein the outer container encloses the inner bag and is filled with a propellant compressing the inner bag, and a valve mechanism attached to the inner bag moveable between an open position, in which a composition stored in the inner bag is allowed to be discharged by the pressure of the compressed gas in foam form, and a closed position, in which the composition is not allowed to be discharged. The choice and difference of pressure within the bag and container enable to have selectively instantaneous or delayed foam for the same composition within the bag.

Such gradual foaming is, for example, that it takes for at least about 10 seconds, preferably at least about 20 seconds, more preferably at least about 30 seconds for the composition to be completely foamed. In such gradual foaming, the composition is dispensed from the package as cream, and then gradually foaming Such gradual foaming may provide easiness to control the application amount, and/or easiness to apply on hair as it may spreads as creams then turning into foams which may increase the contact with hairs at surface areas of hair bundles and also insides of hair bundles.

### Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions.

These include, for example, conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; coloring agents, such as any of the FD&C or D&C dyes; perfumes; ultraviolet and infrared screening and absorbing agents such as benzophenones; and antidandruff agents such as zinc pyrithione.

### Product Forms

The compositions of the present invention can be in the form of rinse-off products or leave-on products, preferably rinse-off products. The hair care compositions of the present invention can be used as a wide variety of hair care products, including but not limited to hair conditioning products, hair treatment products, and hair styling products.

The composition of the present invention is especially suitable for rinse-off hair conditioner. Such compositions are preferably used by following steps:

(i) after shampooing hair, applying to the hair an effective amount of the conditioning compositions for conditioning the hair; and
(ii) then rinsing the hair.

### Key Features of the Invention

A. The present invention is directed to a hair care composition comprising:

a high melting point fatty compound;

a cationic surfactant system;

an aqueous carrier; and

a propellant; and

wherein the composition has a foam density of at least about 0.3, and wherein the composition has a foam collapse resistance value of at least about 1.5 kg·m·s⁻² [N].
B. The composition of the preceding feature A, wherein the composition has the foam density of at least about 0.3 for at least about 10 seconds, preferably the foam density of at least about 0.3 for at least about 20 seconds, more preferably the foam density of at least about 0.3 for at least about 30 seconds.
C. The composition of any of the preceding features, wherein the foam collapse resistance value upon compression is at least from about 1.5 kg·m·s⁻² [N] to about 6.0 kg·m·s⁻² [N], preferably at least from about 1.7 kg·m·s⁻²[N] to about 5.5 kg·m·s⁻²[N], more preferably at least from about 1.9 kg·m·s⁻²[N] to about 5.0 kg·m·s⁻² [N].
D. The composition of any of the preceding features, comprising by weight of the composition, from about 1.5% to about 8%, preferably from about 2.5% to about 7%, more preferably from about 3.0% to about 6% of the one or more high melting point fatty compounds.
E. The composition of any of the preceding features, wherein the weight ratio of the cationic surfactant system to the high melting point fatty compound is from about 1:1 to about 1:10, preferably from about 1:2 to about 1:6, more preferably from about 1:2 to about 1:4.
F. The composition of any of the preceding features, wherein the total amount in mol of the cationic surfactant system over the combination of cationic surfactant system combined with the high melting point fatty compounds is from about 18% to about 30%, preferably from about 22% to about 28%, more preferably from about 24% to about 27%.
G. The composition of any of the preceding features, comprising by weight of the composition, from about 2% to about 10%, preferably from about 2.5% to about 5.5% of the propellant.
H. The composition of any of the preceding features, further comprising a mixture of a soy oligomer and soybean oil.
I. The composition of any of the preceding features, further comprising an anionic polymer comprising a vinyl monomer (A) with a carboxyl group, wherein the vinyl monomer (A) is contained in the polymer at a level of from about 10 mass % to about 90 mass % based on the total mass of the anionic polymer.
J. The composition of the preceding feature I, wherein the anionic polymer further comprises a vinyl monomer (B) expressed by the following formula (1);

CH₂═C(R¹)—CO—X-(Q-O)ᵣ—R² (1)

wherein: R¹ represents a hydrogen atom or a methyl group; R² represents a hydrogen atom or an alkyl group with from 1 to 5 carbon atoms, which may have a substitution group; Q represents an alkylene group with from 2 to 4 carbon atoms which may also have a substitution group; r represents an integer from 2 to 15; and X represents an oxygen atom or an NH group; and, in the following structure -(Q-O)ᵣ—R², the number of atoms bonded in a straight chain is 70 or less.

### Examples

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

TABLE 1
Compositions (wt %)

| Components | Ex. 1 | Ex. 2 | Ex. 3 | Ex. 4 | Ex. 5 | Ex. 6 | Ex. 7 | Ex. 8 | CEx.i |
|---|---|---|---|---|---|---|---|---|---|
| Stearamidopropyl | 1.13 | — | 1.13 | — | 1.6 | — | 1.06 | 1.94 | — |
| dimethyl amine | | | | | | | | | |
| L-glutamic acid | 0.37 | — | 0.37 | — | 0.5 | — | — | 0.63 | — |
| Behenyl | — | 1.48 | — | 4.23 | 2.1 | 2.85 | — | — | — |
| trimethylammonium | | | | | | | | | |
| methosulfate | | | | | | | | | |
| Dicetyldimonium | — | 0.50 | — | — | — | — | 0.50 | — | 0.79 |
| chloride | | | | | | | | | |
| Cetyl alcohol | 1.17 | 0.93 | 1.17 | 3.10 | 3.8 | 1.0 | 1.54 | 1.68 | 2.84 |
| Stearyl alcohol | 2.0 | 2.32 | 2.0 | 2.14 | 2.9 | 2.52 | 2.84 | 2.90 | |
| Glycerin | 50 | — | — | — | — | — | — | — | 0.97 |
| Silicone | 0.35 | 1.0 | 0.35 | 3.0 | 3.5 | — | — | 0.5 | 0.3 |
| Anionic | — | — | — | — | — | 0.5 | — | — | — |
| deposition | | | | | | | | | |
| polymer | | | | | | | | | |
| Soy oligomer and | — | — | — | — | — | — | 0.5 | — | — |
| soybean oil | | | | | | | | | |
| Preservatives | 0.9 | 0.5 | 0.9 | 0.5 | 0.5 | 0.5 | 0.5 | 0.5 | 0.05 |
| Perfume | 0.4 | 0.5 | 0.4 | 0.5 | 0.5 | 0.5 | 0.5 | 0.5 | unknown |
| Propellant | 3 | 3 | 3 | 5 | 5 | 3 | 3 | 3 | 1.1 |
| Deionized Water | q.s. to 100% | | | | | | | | |
| Foam density | 0.8 at | — | — | — | — | 0.8 at | 0.3 as | 0.5 as | 0.1 as |
| | least | | | | | least | dispensed | dispensed | dispensed |
| | for | | | | | for | | | |
| | 20 sec. | | | | | 10 sec. | | | |
| Foam collapse | 6.5 | — | — | — | — | 4.5 | 3.4 | 4.2 | 0.8 |
| resistance value | | | | | | | | | |

### Method of Preparation

The hair care compositions of “Ex. 1” through “Ex.8” of the present invention and the hair care composition of “CEx.i” as a comparative example, as shown above, can be prepared by any conventional method well known in the art, and dispensed from any conventional package well known in the art.

### Properties and Benefits

Examples 1 through 8 are hair care compositions of the present invention, which are particularly useful as rinse-off hair conditioning compositions. After shampooing hair, an effective amount of the hair care compositions are applied to the hair, and then rinsed off.

The embodiments disclosed and represented by the previous “Ex. 1” through “Ex. 8” have the required foam density and foam collapse resistance value, and have many advantages. For example, they provide improved wet conditioning benefits, spreadability, and/or even deposition, compared to a comparative example “CEx. i”.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair care composition comprising:
a high melting point fatty compound;
a cationic surfactant system;
an aqueous carrier; and
a propellant; and
wherein the composition has a foam density of at least about 0.3, and wherein the composition has a foam collapse resistance value of at least about 1.5 kg·m·s⁻² [N].

2. The composition of claim 1, wherein the composition has the foam density of at least about 0.3 for at least about 10 seconds.

3. The composition of claim 2, wherein the composition has the foam density of at least about 0.3 for at least about 20 seconds.

4. The composition of claim 3, wherein the composition has the foam density of at least about 0.3 for at least about 30 seconds.

5. The composition of claim 1, wherein the foam collapse resistance value upon compression is at least from about 1.5 kg·m·s⁻² [N] to about 6.0 kg·m·s⁻² [N].

6. The composition of claim 5, wherein the foam collapse resistance value is at least from about 1.7 kg·m·s⁻² [N] to about 5.5 kg·m·s⁻² [N].

7. The composition of claim 6, wherein the foam collapse resistance value is at least from about 1.9 kg·m·s⁻² [N] to about 5.0 kg·m·s⁻² [N].

8. The composition of claim 1, comprising by weight of the composition, from about 1.5% to about 8% of the one or more high melting point fatty compounds.

9. The composition of claim 8, comprising by weight of the composition, from about 2.5% to about 7% of the one or more high melting point fatty compounds.

10. The composition of claim 9, comprising by weight of the composition, from about 3.0% to about 6% of the one or more high melting point fatty compounds.

11. The composition of claim 1, wherein the weight ratio of the cationic surfactant system to the high melting point fatty compound is from about 1:1 to about 1:10.

12. The composition of claim 11, wherein the weight ratio of the cationic surfactant system to the high melting point fatty compound is from about 1:2 to about 1:6.

13. The composition of claim 12, wherein the weight ratio of the cationic surfactant system to the high melting point fatty compound is from about 1:2 to about 1:4.

14. The composition of claim 1, wherein the total amount in mol of the cationic surfactant system over the combination of cationic surfactant system combined with the high melting point fatty compounds is from about 18% to about 30%.

15. The composition of claim 14, wherein the total amount in mol of the cationic surfactant system over the combination of cationic surfactant system combined with the high melting point fatty compounds is from about 22% to about 28%.

16. The composition of claim 15, wherein the total amount in mol of the cationic surfactant system over the combination of cationic surfactant system combined with the high melting point fatty compounds is from about 24% to about 27%.

17. The composition of claim 1, comprising by weight of the composition, from about 2% to about 10% of the propellant.

18. The composition of claim 17, comprising by weight of the composition, from about 2.5% to about 5.5% of the propellant.

19. The composition of claim 1, further comprising a mixture of a soy oligomer and soybean oil.

20. The composition of claim 1, further comprising an anionic polymer comprising a vinyl monomer (A) with a carboxyl group, wherein the vinyl monomer (A) is contained in the polymer at a level of from about 10 mass % to about 90 mass % based on the total mass of the anionic polymer.

21. The composition of claim 20, wherein the anionic polymer further comprises a vinyl monomer (B) expressed by the following formula (1):
CH₂═C(R¹)—CO—X-(Q-O)ᵣ—R² (1)
wherein: R¹ represents a hydrogen atom or a methyl group; R² represents a hydrogen atom or an alkyl group with from 1 to 5 carbon atoms, which may have a substitution group; Q represents an alkylene group with from 2 to 4 carbon atoms which may also have a substitution group; r represents an integer from 2 to 15; and X represents an oxygen atom or an NH group; and, in the following structure -(Q-O)ᵣ—R², the number of atoms bonded in a straight chain is 70 or less.
